SEQUENCE LISTING (4 sequences)

SEQ ID NO: 1 - DNA, 22 bp, Artificial Sequence
  Forward primer
aaagcgtccaggccaccacaga

SEQ ID NO: 2 - DNA, 23 bp, Artificial Sequence
  reverse primer
ggcagaaaatggcacacggttac

SEQ ID NO: 3 - DNA, 20 bp, Artificial Sequence
  forward primer
agaaaatctggcaccacacc

SEQ ID NO: 4 - DNA, 20 bp, Artificial Sequence
  reverse primer
aacggcagaagagagaacca

(19) United States
(12) Patent Application Publication
(10) Pub. No.: US 2023/0355557 A1
(43) Pub. Date: Nov. 9, 2023

(54) ANTIVIRAL SOX INHIBITORS

(71) Applicant: Gertrude Biomedical Pty LTD, Victoria (AU)

(72) Inventors: Mathias Frederic FRANCOIS, New South Wales (AU); Johannes ZUEGG, Queensland (AU)

(21) Appl. No.: 18/023,121

(22) PCT Filed: Aug. 26, 2020
(86) PCT No.: PCT/AU2020/050892

§ 371 Date: Feb. 24, 2023

(51) Int. Cl. A61P 31/22; A61K 31/235; A61K 31/192

(52) U.S. Cl. CPC A61K 31/192; A61K 31/235; A61P 31/22

(57) ABSTRACT

The present disclosure generally relates to antiviral compounds for use in treatment of viral associated diseases or conditions. The present disclosure also relates to processes for preparing the antiviral compounds, and uses or methods of treatment of viral associated diseases or conditions comprising the antiviral compounds. The present disclosure also provides antiviral compounds as inhibitors of SOX family transcription factors, and in particular 5 SOX18 transcription factor. In particular, the antiviral compounds are based on a biaryl benzoic acid scaffold according to Formula 1 as described herein.

### FIELD

The present disclosure generally relates to antiviral compounds for use in treatment of viral associated diseases or conditions. The present disclosure also relates to processes for preparing the antiviral compounds, and uses or methods of treatment of viral associated diseases or conditions comprising the antiviral compounds. The present disclosure also provides antiviral compounds as inhibitors of SOX family transcription factors, and in particular SOX18 transcription factor.

### BACKGROUND

Kaposi sarcoma (KS) is an angiogenic endothelial tumour caused by KS herpesvirus (KSHV). Most cases of KS develop in people infected with HIV, such patients having a 20,000-fold increased risk of developing KS compared to people without HIV. KSHV infection is much more common in some parts of the world, such as subequatorial Africa, where over 30% of the population carries KSHV antibodies. In some areas in Africa, the virus seems to spread from mother to child. Seropositivity for the virus ranges from 10% to 25% in the Mediterranean area. In other regions of the world where KSHV is not endemic, the seroprevalence is around 2-5%. (Horenstein et al., (2008); J. Cutan. Pathol. 35(Suppl. 2): 40-44).

KS cells form purple, brown or red lesions on the skin that are usually papular (i.e, palpable or raised). In many cases, these skin lesions do not cause any symptoms; in other cases, they may cause painful swelling, especially in the legs, groin area or skin around the eyes. KS can cause serious problems, and can even become life-threatening when the lesions are in the lungs, liver or digestive tracts. Lesions in the digestive tract may cause blockage, resulting in nausea, vomiting, abdominal pain and occasionally bleeding. Lesions in the lungs can cause difficulty breathing.

KSHV appears to be transmitted through saliva, as is the case for other human herpesviruses. Sexual transmission through semen has also been suggested (Horenstein et al., (2008); J. Cutan. Pathol. 35(Suppl. 2): 40-44). The virus may also be transmitted through organ donation. Some cases of KSHV have been reported in injection drug users and are thought to be spread when needles are contaminated with infected blood. The transmission of KSHV through blood appears to be rare and occurs much less than HIV transmission.

The histopathological hallmark of KS is the presence of KSHV-positive spindle cells (SC), the tumour cells of KS (Ojala, P. M. & Schulz, T. F. (2014) Semin Cancer Biol 26:69-77; Gramolelli, S. & Ojala, P. M. (2017) Curr Opin Virol 26:156-162). The cell of origin of SC has been debated for two decades. The prevailing hypothesis suggests lymphatic endothelial origin, although blood endothelial cells or mesenchymal cells are also candidates (Li, Y. et al. (2018) Cancer Res 78:230-245).

In vitro, latency is the default replication program in KSHV-infected cells with undetectable levels of lytic genes expressed. However, KSHV infection of lymphatic, but not blood, endothelial cells (LEC and BEC) leads to a unique infection program characterized by high KSHV genome copies, spontaneous lytic gene expression and release of infectious virus.

During embryonic development, LEC precursors originate from COUPTF2/SOX18 double-positive BECs that physically separate from the cardinal vein to establish a primary lymphatic vascular plexus. In this process, COUPTF2 and SOX18 drive the expression of PROX1 thereby orchestrating LEC differentiation (Francois, M. et al. (2008) Nature 822:456); Srinivasan, R. S. et al. (2010) Genes Dev 24, 696-707).

There is a need for alternatives to anti-viral drug approaches for treating viruses such as KSHV.

It will be understood that any prior art publications referred to herein do not constitute an admission that any of these documents form part of the common general knowledge in the art, in Australia or in any other country.

### SUMMARY

The present disclosure is based on a finding that SOX18 binds to viral origins of replication and increases viral genome copies. Accordingly, the inventors have undertaken an extensive development project to identify SOX inhibitors, and in particular SOX18 inhibitors, for an ability to inhibit viral replication, which has involved using Kaposi sarcoma as an example.

In one aspect, there is provided a method of treating a viral disease or condition by administration of an antiviral compound to a subject in need of treatment thereof, wherein the antiviral compound is a compound of Formula 1:

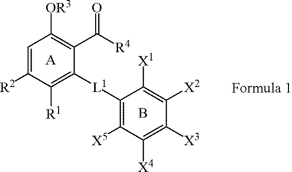

wherein
R¹ and R² are each independently selected from hydrogen, halo, OH, C₁₋₁₀alkyl, C₁₋₁₀alkylhalo, OC₁₋₁₀alkyl, OC₁₋₁₀alkylhalo, C₁₋₁₀alkenyl, C₁₋₁₀alkenylhalo, OC₁₋₁₀alkenyl, OC₁₋₁₀alkenylhalo;
R³ is selected from hydrogen, C₁₋₁₀alkyl, C₁₋₁₀alkylhalo, C₁₋₁₀alkenyl, C₁₋₁₀alkenylhalo;
R⁴ is selected from hydrogen, OH, C₁₋₁₀alkyl, OC₁₋₁₀alkyl, C₁₋₁₀alkylhalo, OC₁₋₁₀alkylhalo, NH₂, NH(C₁₋₁₀alkyl), and N(C₁₋₁₀alkyl)₂;
L¹ is selected from C₁₋₁₀alkyl, OC₁₋₁₀alkyl, C₁₋₁₀alkenyl, OC₁₋₁₀alkenyl, OC(═O), OC(=O)(C₁₋₁₀alkyl), NHC(═O), N(C₁₋₁₀alkyl)C(=O), OS(═O)₂, wherein each alkyl or alkenyl is uninterrupted or interrupted with one or more groups selected from O, OC(═O), NH, N(C₁₋₁₀alkyl), NHC(═O), S, and S(═O)₂, and unsubstituted or substituted with one or more groups selected from halo, OH, and C═O; and
X¹, X², X³, X⁴, and X⁵, are each independently selected from hydrogen, halo, OH, C₁₋₁₀alkyl, C₁₋₁₀alkylhalo, OC₁₋₁₀alkyl, OC₁₋₁₀alkylhalo, C₁₋₁₀alkenyl, C₁₋₁₀alkenylhalo, OC₁₋₁₀alkenyl, OC₁₋₁₀alkenylhalo, C(═O)H, C(═O)OH, C(=O)O(C₁₋₁₀alkyl), and NO₂, and any two X groups can join together to form an aryl group unsubstituted or substituted with one or more groups selected from halo, OH, C₁₋₁₀alkyl, C₁₋₁₀alkylhalo, OC₁₋₁₀alkyl, OC₁₋₁₀alkylhalo, C₁₋₁₀alkenyl, C₁₋₁₀alkenylhalo, OC₁₋₁₀alkenyl, OC₁₋₁₀alkenylhalo, C(═O)H, C(═O)OH, C(=O)O(C₁₋₁₀alkyl), and NO₂.

In another aspect, there is provided a method of treating a viral disease or condition in a subject comprising administering an antiviral compound to a subject in need of treatment thereof, wherein the antiviral compound is a compound of Formula 1, or pharmaceutically acceptable salt, solvate, or stereoisomer thereof.

In another aspect, there is provided a use of a compound of Formula 1 as defined according to any aspects, embodiments or examples as described herein, as an antiviral agent or for treating a viral disease or condition or a viral associated disease or condition.

In another aspect, there is provided a use of a compound of Formula 1 as defined according to any aspects, embodiments or examples as described herein, in the manufacture of a medicament for treating a viral disease or condition or a viral associated disease or condition.

In another aspect, there is provided a method for inhibiting replication of a herpesvirus and/or treating a viral disease or condition caused by a herpesvirus in a subject comprising administering to the subject a compound of Formula 1 as described herein

It will be appreciated that other aspects, embodiments and examples of the compounds, pharmaceutical compositions, methods, or uses, are further described herein.

### DESCRIPTION OF THE DRAWINGS

FIG. 1 shows (a,b) luciferase reporter assays in HeLa cells transfected as indicated with increasing amounts of SOX18. Single values from (b) n=8 and (c) n=4 biological replicates are shown. Bars represent mean ± SEM.

FIG. 2 shows HeLa cells transfected for 18 hours with SOX18 and one of two luciferase reporter constructs: 7XTR and OriA before being treated with GB001 (SM4), GB002 or GB004 for 24 hours. Luciferase signal was quantified and single values from n=3 technical replicates are shown. Data points represent mean + SD. P values were calculated using a 2-way and ordinary one way ANOVA followed by Dunnett’s multiple comparison test.

FIG. 3 shows LECs infected with KSHV and treated with compounds for 6 days at the concentrations shown. The relative expression of KSHV from control was quantified and single values from n=3 technical replicates are shown. Bars represent mean + SD. P values were calculated using a 2-way ANOVA (left) and ordinary one way-ANOVA (right) followed by Dunnett’s multiple comparison test.

### KEY TO SEQUENCE LISTING

SEQ ID NO:1 sequence of the K8.1 forward primer
SEQ ID NO:2 sequence of the K8.1 reverse primer
SEQ ID NO:3 sequence of the genomic forward primer
SEQ ID NO:4 sequence of the genomic reverse primer

### DETAILED DESCRIPTION

### General Definitions

The following definitions apply to the terms as used throughout this specification, unless otherwise limited in specific instances. Unless specifically defined otherwise, all technical and scientific terms used herein shall be taken to have the same meaning as commonly understood by one of ordinary skill in the art.

As used herein, the term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/- 20%, more preferably +/- 10%, of the designated value.

As used herein, singular forms “a”, “an” and “the” include plural aspects, unless the context clearly indicates otherwise.

Throughout this specification, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “subject” refers to any organism susceptible to a disease or condition. For example, the subject can be a mammal, primate, livestock (e.g., sheep, cow, horse, pig), companion animal (e.g., dog, cat), or laboratory animal (e.g., mouse, rabbit, rat, guinea pig, hamster). In one example, the subject is a mammal. In one embodiment, the subject is human. In one embodiment, the disease or condition is associated with a virus.

As used herein, the term “treating” includes alleviation or reducing symptoms associated with a specific disorder or condition.

As used herein, the term “prevention” includes prophylaxis of the specific disorder or condition. For example, as used herein, the term “preventing” refers to preventing the onset or duration of the symptoms associated with a virus.

The present disclosure relates to compounds of Formula 1 and salts thereof. Salts may be formed in the case of embodiments of the compound of Formula 1 which contain a suitable acidic or basic group. Suitable salts of the compound of Formula 1 include those formed with organic or inorganic acids or bases.

As used herein, the phrase “pharmaceutically acceptable salt” or like term refers to pharmaceutically acceptable organic or inorganic salts. It will be appreciated that any reference to “salt” herein can include “pharmaceutically acceptable salts”. Exemplary acid addition salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. Exemplary base addition salts include, but are not limited to, ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl -propylamine, or a mono-, di- or trihydroxy lower alkylamine, for example mono-, di- or triethanolamine. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion. It will also be appreciated that non-pharmaceutically acceptable salts also fall within the scope of the present disclosure since these may be useful as intermediates in the preparation of pharmaceutically acceptable salts or may be useful during storage or transport.

Those skilled in the art of organic and/or medicinal chemistry will appreciate that many organic compounds can form complexes with solvents in which they are reacted or from which they are precipitated or crystallized. These complexes are known as “solvates”. For example, a complex with water is known as a “hydrate”. As used herein, the phrase “pharmaceutically acceptable solvate” or “solvate” refer to an association of one or more solvent molecules and a compound of the present disclosure. Examples of solvents that form pharmaceutically acceptable solvates include, but are not limited to, water, isopropanol, ethanol, methanol, DMSO, ethyl acetate, acetic acid, and ethanolamine. It will be understood that the present disclosure encompasses solvated forms, including hydrates, of the compounds of formula (I) and salts thereof.

The compounds of the present disclosure may contain chiral (asymmetric) centers or the molecule as a whole may be chiral. The individual stereoisomers (enantiomers and diastereoisomers) and mixtures of these are within the scope of the present disclosure.

As used herein, the term “stereoisomer” refers to compounds having the same molecular formula and sequence of bonded atoms (i.e., atom connectivity), though differ in the three-dimensional orientations of their atoms in space. As used herein, the term “enantiomers” refers to two compounds that are stereoisomers in that they are non-superimposable mirror images of one another. Relevant stereocenters may be donated with (R)- or (S)- configuration.

Those skilled in the art of organic and/or medicinal chemistry will appreciate that the compounds of Formula 1 and salts thereof may be present in amorphous form, or in a crystalline form. It will be understood that the present disclosure encompasses all forms and polymorphs of the compounds of Formula 1 and salts thereof.

As would be understood by the person skilled in the art, a compound of Formula 1, or any salt, solvate or stereoisomer thereof would be administered in a therapeutically effective amount. The term “therapeutically effective amount”, as used herein, refers to a compound being administered in an amount sufficient to alleviate or prevent to some extent one or more of the symptoms of the disorder or condition being treated. The result can be the reduction and/or alleviation of the signs, symptoms, or causes of a disease or condition, or any other desired alteration of a biological system. In one embodiment, the term “therapeutically effective amount” refers to a compound of Formula 1, or any salt thereof, being administered in an amount sufficient to inhibit or modulate a SOX transcription factor (e.g. SOX18) to provide a therapeutic outcome.

As used herein, the term “halogen” or “halo” means fluorine, chorine, bromine, or iodine.

As used herein, the term “alkyl” encompasses both straight chain (i.e. linear) and branched chain hydrocarbon groups. Examples of alkyl groups include methyl, ethyl, n-propyl, iso-propyl, n-butyl, t-butyl, i-butyl, sec-butyl, pentyl, and hexyl groups. In one example, the alkyl group is of 1 to 20 carbon atoms (i.e. C₁₋₂₀alkyl). In another example, the alkyl group is of 1 to 10 carbon atoms (i.e. C₁₋₁₀alkyl). In another example, the alkyl group is 2 to 10 carbon atoms (i.e. C₂₋₁₀alkyl). In another example, the alkyl group is 1 to 6 carbon atoms (i.e. C₁₋₆alkyl) or 2 to 6 carbon atoms (i.e. C₂₋₆alkyl). In another example, the alkyl group is 1 to 4 carbon atoms (i.e. C₁₋₄alkyl) or 2 to 4 carbon atoms (i.e. C₂₋₄alkyl).

As used herein, the term “alkenyl” refers to both straight and branched chain unsaturated hydrocarbon groups with at least one carbon-carbon double bond. Examples of alkenyl groups include ethenyl, propenyl, butenyl, pentenyl, and hexenyl groups. In one example, the alkenyl group is of 1 to 20 carbon atoms (i.e. C₁₋₂₀alkenyl). In another example, the alkenyl group is of 1 to 10 carbon atoms (i.e. C₁₋₁₀alkenyl). In another example, the alkenyl group is 2 to 10 carbon atoms (i.e. C₁₋₁₀alkenyl). In another example, the alkenyl group is 1 to 6 carbon atoms (i.e. C₁₋₆alkenyl) or 2 to 6 carbon atoms (i.e. C₂₋₆alkenyl). In another example, the alkenyl group is 1 to 4 carbon atoms (i.e. C₁₋₄alkenyl) or 2 to 4 carbon atoms (i.e. C₂₋₄alkenyl).

As used herein, the term “alkynyl” refers to both straight and branched chain unsatu-rated hydrocarbon groups with at least one carbon-carbon triple bond. Examples of alkynyl groups include ethynyl, propynyl, butynyl, pentynyl, and hexynyl groups. In one example, the alkynyl group is of 1 to 20 carbon atoms (i.e. C₁₋₂₀alkynyl). In another example, the alkynyl group is of 1 to 10 carbon atoms (i.e. C₁₋₁₀alkynyl). In another example, the alkynyl group is 2 to 10 carbon atoms (i.e. C₁₋₁₀alkynyl). In another example, the alkynyl group is 1 to 6 carbon atoms (i.e. C₁₋₆alkynyl) or 2 to 6 carbon atoms (i.e. C₂₋₆alkynyl). In another example, the alkynyl group is 1 to 4 carbon atoms (i.e. C₁₋₄alkynyl) or 2 to 4 carbon atoms (i.e. C₂₋₄alkynyl).

As used herein, the term “alkylhalo” refers to an alkyl group having at least one halogen substituent, where “alkyl” and “halogen” are as described above. Examples of alkylhalo groups include fluoromethyl, chloromethyl, bromomethyl, iodomethyl, fluoropropyl, and fluorobutyl groups. Examples include difluoromethyl and difluoroethyl groups, and trifluoromethyl and trifluoroethyl groups.

As used herein, the term “alkenylhalo” refers to an alkenyl group having at least one halogen substituent, where “alkenyl” and “halogen” are as described above.

As used herein, the term “alkynylhalo” refers to an alkynyl group having at least one halogen substituent, where “alkynyl” and “halogen” are as described above.

“Aryl” whether used alone, or in compound words, such as arylalkyl, represents: (i) an optionally substituted mono- or polycyclic aromatic carbocyclic moiety, e.g., of about 6 to about 20 carbon atoms, such as phenyl, naphthyl or fluorenyl; or, (ii) an optionally substituted partially saturated polycyclic carbocyclic aromatic ring system in which an aryl and a cycloalkyl or cycloalkenyl group are fused together to form a cyclic structure such as a tetrahydronaphthyl, indenyl, indanyl or fluorene ring. It will be appreciated that the polycyclic ring system includes bicyclic and tricyclic ring systems. In further examples the term “aryl” denotes single, polynuclear, conjugated and fused residues of aromatic hydrocarbons, such as unsubstituted or substituted: phenyl, biphenyl, terphenyl, quaterphenyl, phenoxyphenyl, naphthyl, tetrahydronaphthyl, anthracenyl, dihydroanthracenyl, benzanthracenyl, dibenzanthracenyl and phenanthrenyl groups.

As used herein, the term “alkylaryl” refers to an alkyl group interrupted and/or substituted with at least one aryl group, where “alkyl” and “aryl” are as described above.

As used herein, the term “saturated” refers to a group where all available valence bonds of the backbone atoms are attached to other atoms Representative examples of saturated groups include, but are not limited to, butyl, cyclohexyl, piperidine, and the like.

As used herein, the term “unsaturated” refers to a group where at least one valence bond of two adjacent backbone atoms is not attached to other atoms. Representative examples include, but are not limited to, alkenes (e.g., —CH₂—CH═CH—), phenyl, pyrrole, and the like.

As used herein, the term “substituted” refers to a group having one or more hydrogens or other atoms removed from a carbon or suitable heteroatom and replaced with a further group (i.e., substituent).

As used herein, the term “unsubstituted” refers to a group that does not have any further groups attached thereto or substituted therefore.

All documents cited or referenced herein, and all documents cited or referenced in herein cited documents, together with any manufacturer’s instructions, descriptions, product specifications, and product sheets for any products mentioned herein or in any document incorporated by reference herein, are hereby incorporated herein by reference in their entirety.

### Compounds of Formula 1

The present disclosure is directed to providing antiviral compounds. The antiviral compounds can be effective inhibitors of one or more SOX family transcription factors, and in particular SOX18. It will be appreciated that the SOX family transcription factors are sex determining region Y proteins (SRY-related HMG-box proteins). The antiviral compounds are based on a benzoic acid scaffold or derivative, and in particular a biaryl benzoic acid scaffold. The antiviral compounds of the present disclosure can be provided by a compound of Formula 1 as described herein.

In one aspect, compounds of Formula 1 can be provided as follows:

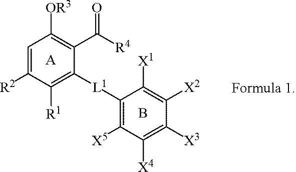

In one example of the above Formula 1:
R¹ and R² are each independently selected from hydrogen, halo, OH, C₁₋₁₀alkyl, C₁₋₁₀alkylhalo, OC₁₋₁₀alkyl, OC₁₋₁₀alkylhalo, C₁₋₁₀alkenyl, C₁₋₁₀alkenylhalo, OC₁₋₁₀alkenyl, OC₁₋₁₀alkenylhalo;
R³ is selected from hydrogen, C₁₋₁₀alkyl, C₁₋₁₀alkylhalo, C₁₋₁₀alkenyl, C₁₋₁₀alkenylhalo;
R⁴ is selected from hydrogen, OH, C₁₋₁₀alkyl, OC₁₋₁₀alkyl, C₁₋₁₀alkylhalo, OC₁₋₁₀alkylhalo, NH₂, NH(C₁₋₁₀alkyl), and N(C₁₋₁₀alkyl)₂;
L¹ is selected from C₁₋₁₀alkyl, OC₁₋₁₀alkyl, C₁₋₁₀alkenyl, OC₁₋₁₀alkenyl, OC(═O), OC(=O)(C₁₋₁₀alkyl), NHC(═O), N(C₁₋₁₀alkyl)C(=O), OS(═O)₂, wherein each alkyl or alkenyl is uninterrupted or interrupted with one or more groups selected from O, OC(═O), NH, N(C₁₋₁₀alkyl), NHC(═O), S, and S(═O)₂, and unsubstituted or substituted with one or more groups selected from halo, OH, and C═O; and
X¹, X², X³, X⁴, and X⁵, are each independently selected from hydrogen, halo, OH, C₁₋₁₀alkyl, C₁₋₁₀alkylhalo, OC₁₋₁₀alkyl, OC₁₋₁₀alkylhalo, C₁₋₁₀alkenyl, C₁₋₁₀alkenylhalo, OC₁₋₁₀alkenyl, OC₁₋₁₀alkenylhalo, C(═O)H, C(═O)OH, C(=O)O(C₁₋₁₀alkyl), and NO₂, and any two X groups can join together to form an aryl group unsubstituted or substituted with one or more groups selected from halo, OH, C₁₋₁₀alkyl, C₁₋₁₀alkylhalo, OC₁₋₁₀alkyl, OC₁₋₁₀alkylhalo, C₁₋₁₀alkenyl, C₁₋₁₀alkenylhalo, OC₁₋₁₀alkenyl, OC₁₋₁₀alkenylhalo, C(═O)H, C(═O)OH, C(=O)O(C₁₋₁₀alkyl), and NO₂.

In another example of the above Formula 1:
R¹ and R² are each independently selected from hydrogen, halo, OH, C₁₋₁₀alkyl, C₁₋₁₀alkylhalo, OC₁₋₁₀alkyl, and OC₁₋₁₀alkylhalo;
R³ is selected from hydrogen, C₁₋₁₀alkyl and C₁₋₁₀alkylhalo;
R⁴ is selected from OH, OC₁₋₁₀alkyl, and OC₁₋₁₀alkylhalo;
L¹ is selected from C₁₋₁₀alkyl, OC₁₋₁₀alkyl, C₁₋₁₀alkenyl, and OC₁₋₁₀alkenyl, wherein each alkyl or alkenyl is uninterrupted or interrupted with one or more groups selected from O, OC(═O), NH, N(C₁₋₁₀alkyl), NHC(═O), S, and S(═O)₂, and unsubstituted or substituted with one or more groups selected from halo, OH, and C═O; and
X¹, X², X³, X⁴, and X⁵, are each independently selected from hydrogen, halo, OH, C₁₋₁₀alkyl, C₁₋₁₀alkylhalo, OC₁₋₁₀alkyl, OC₁₋₁₀alkylhalo, and any two X groups can join together to form an aryl group unsubstituted or substituted with one or more groups selected from halo, OH, C₁₋₁₀alkyl, C₁₋₁₀alkylhalo, OC₁₋₁₀alkyl, OC₁₋₁₀alkylhalo.

In another example of the above Formula 1:
R¹ and R² are each independently selected from hydrogen, halo, OH, C₁₋₁₀alkyl, C₁₋₁₀alkylhalo, OC₁₋₁₀alkyl, and OC₁₋₁₀alkylhalo;
R³ is selected from hydrogen, C₁₋₁₀alkyl and C₁₋₁₀alkylhalo;
R⁴ is selected from OH, OC₁₋₁₀alkyl, and OC₁₋₁₀alkylhalo;
L¹ is selected from C₂₋₄alkyl, OC₂₋₄alkyl, C₂₋₄alkenyl, and OC₂₋₄alkenyl, wherein each alkyl or alkenyl is uninterrupted or interrupted with one or more groups selected from O, OC(═O), NH, N(C₁₋₁₀alkyl), NHC(═O), S, and S(═O)₂, and unsubstituted or substituted with one or more groups selected from halo, OH, and C═O; and
X¹, X², X³, X⁴, and X⁵, are each independently selected from hydrogen, halo, OH, C₁₋₁₀alkyl, C₁₋₁₀alkylhalo, OC₁₋₁₀alkyl, OC₁₋₁₀alkylhalo, and any two X groups can join together to form an aryl group unsubstituted or substituted with one or more groups selected from halo, OH, C₁₋₁₀alkyl, C₁₋₁₀alkylhalo, OC₁₋₁₀alkyl, OC₁₋₁₀alkylhalo.

In another example of the above Formula 1:
R¹ and R² are each independently selected from hydrogen, halo, OH, C₁₋₁₀alkyl, C₁₋₁₀alkylhalo, OC₁₋₁₀alkyl, and OC₁₋₁₀alkylhalo;
R³ is selected from hydrogen, C₁₋₁₀alkyl and C₁₋₁₀alkylhalo;
R⁴ is selected from OH, OC₁₋₁₀alkyl, and OC₁₋₁₀alkylhalo;
L¹ is selected from C₂₋₄alkyl, OC₂₋₄alkyl, C₂₋₄alkenyl, and OC₂₋₄alkenyl, wherein each alkyl or alkenyl is uninterrupted or interrupted with one or more groups selected from O, OC(═O), NH, N(C₁₋₁₀alkyl), NHC(═O), S, and S(═O)₂, and unsubstituted or substituted with one or more groups selected from halo, OH, and C═O; and
X¹, X², X³, X⁴, and X⁵, are each independently selected from hydrogen, halo, OH, C₁₋₁₀alkyl, C₁₋₁₀alkylhalo, OC₁₋₁₀alkyl, OC₁₋₁₀alkylhalo, and any two X groups can join together to form a monocyclic or bicyclic aryl group unsubstituted or substituted with one or more groups selected from halo, OH, C₁₋₁₀alkyl, C₁₋₁₀alkylhalo, OC₁₋₁₀alkyl, OC₁₋₁₀alkylhalo.

In another example of the above Formula 1:
R¹ and R² are each independently selected from hydrogen and halo;
R³ is selected from hydrogen, C₁₋₁₀alkyl and C₁₋₁₀alkylhalo;
R⁴ is selected from OH, OC₁₋₁₀alkyl, and OC₁₋₁₀alkylhalo;
L¹ is selected from C₂₋₄alkyl, OC₂₋₄alkyl, C₂₋₄alkenyl, and OC₂₋₄alkenyl, wherein each alkyl or alkenyl is uninterrupted or interrupted with one or more groups selected from O, OC(═O), NH, N(C₁₋₁₀alkyl), NHC(═O), S, and S(═O)₂, and unsubstituted or substituted with one or more groups selected from halo, OH, and C═O; and
X¹, X², X³, X⁴, and X⁵, are each independently selected from hydrogen, halo, OH, C₁₋₁₀alkyl, C₁₋₁₀alkylhalo, OC₁₋₁₀alkyl, OC₁₋₁₀alkylhalo, and any two X groups can join together to form a phenyl or naphthyl group unsubstituted or substituted with one or more groups selected from halo, OH, C₁₋₁₀alkyl, C₁₋₁₀alkylhalo, OC₁₋₁₀alkyl, OC₁₋₁₀alkylhalo.

In another example of the above Formula 1:
R¹ and R² are hydrogen;
R³ is hydrogen;
R⁴ is selected from OH and OC₁₋₁₀alkyl;
L¹ is selected from C₂₋₄alkyl and C₂₋₄alkenyl, wherein each is uninterrupted or interrupted with one or more groups selected from O, OC(═O), NH, N(C₁₋₁₀alkyl), NHC(═O), S, and S(═O)₂, and unsubstituted or substituted with one or more groups selected from halo, OH, and C═O; and
X¹, X², X³, X⁴, and X⁵, are each independently selected from hydrogen, halo, OH, C₁₋₁₀alkyl, C₁₋₁₀alkylhalo, OC₁₋₁₀alkyl, OC₁₋₁₀alkylhalo, and any two X groups can join together to form a phenyl group unsubstituted or substituted with one or more groups selected from halo, OH, C₁₋₁₀alkyl, C₁₋₁₀alkylhalo, OC₁₋₁₀alkyl, OC₁₋₁₀alkylhalo.

### A-Ring Groups

The above structure of Formula 1 comprises an aryl “A” ring on the left hand side that is linked via an L¹ group to an aryl “B” ring on the right hand side. A compound of Formula 1 can be provided by a linked biaryl benzoic acid scaffold. In the above formula 1, the A ring groups comprise R¹, R², R³ and R⁴.

R¹ and R² can each be independently selected from hydrogen, halo, OH, C₁₋₁₀alkyl, C₁₋₁₀alkylhalo, OC₁₋₁₀alkyl, OC₁₋₁₀alkylhalo, C₁₋₁₀alkenyl, C₁₋₁₀alkenylhalo, OC₁₋₁₀alkenyl, and OC₁₋₁₀alkenylhalo. In other examples, R¹ and R² can each be independently selected from hydrogen, halo, OH, C₁₋₁₀alkyl, C₁₋₁₀alkylhalo, OC₁₋₁₀alkyl, and OC₁₋₁₀alkylhalo. In other examples of Formula 1, R¹ and R² can each be independently selected from hydrogen, OH, or OC₁₋₁₀alkyl. In other examples of Formula 1, R¹ is hydrogen. In other examples of Formula 1, R¹ and R² are hydrogen.

R³ can be selected from hydrogen, C₁₋₁₀alkyl, C₁₋₁₀alkylhalo, C₁₋₁₀alkenyl, and C₁₋₁₀alkenylhalo. In other examples of Formula 1, R³ is hydrogen or C₁₋₆alkyl. In other examples of Formula 1, R³ is hydrogen.

R⁴ can be selected from hydrogen, OH, C₁₋₁₀alkyl, OC₁₋₁₀alkyl, NH₂, NH(C₁₋₁₀alkyl), and N(C₁₋₁₀alkyl)₂. In other examples of Formula 1, R⁴ is OH or C₁₋₆alkyl. In other examples of Formula 1, R⁴ is OH.

It will be appreciated that the above various definitions and examples of R¹, R², R³ and R⁴ may be combined in any way. For example, in some examples of Formula 1 R¹ is hydrogen, R² is hydrogen, R³ is hydrogen or C₁₋₆alkyl, and R⁴ is OH or OC₁₋₆alkyl.

### Linker Group L¹

The linker group L¹ connects the aryl ring A to the aryl ring B.

L¹ may be selected from C₁₋₁₀alkyl, OC₁₋₁₀alkyl, C₁₋₁₀alkenyl, OC₁₋₁₀alkenyl, OC(═O), OC(=O)(C₁₋₁₀alkyl), NHC(═O), N(C₁₋₁₀alkyl)C(=O), OS(═O)₂. Each alkyl or alkenyl can be uninterrupted or interrupted with one or more groups and/or unsubstituted or substituted with one or more groups. For example, each alkyl or alkenyl is uninterrupted or interrupted with one or more groups selected from O, OC(═O), NH, N(C₁₋₁₀alkyl), NHC(═O), S, and S(═O)₂, and/or unsubstituted or substituted with one or more groups selected from halo, OH, and C═O.

In one example, L¹ may be selected from C₁₋₁₀alkyl or C₁₋₁₀alkenyl. In another example, each alkyl or alkenyl may be uninterrupted or interrupted with one or more groups selected from O, OC(═O), NH, N(C₁₋₁₀alkyl), NHC(═O), S, and S(═O)₂, and/or unsubstituted or substituted with one or more groups selected from halo, OH, and C═O.

In another example, L¹ is selected from C₁₋₆alkyl or C₁₋₆alkenyl. In another example, each alkyl or alkenyl is uninterrupted or interrupted with one or more groups selected from O, NH, NHC(═O), S, and S(═O)₂.

In another example, L¹ is selected from C₂₋₆alkyl or C₂₋₆alkenyl. In another example, each alkyl or alkenyl is uninterrupted or interrupted with one or more groups selected from O, NH, NHC(═O), S, and S(═O)₂.

In another example, L¹ is selected from C₂₋₄alkyl or C₂₋₄alkenyl. In another example, each alkyl or alkenyl is uninterrupted or interrupted with one or more groups selected from O, NH, NHC(═O), S, and S(═O)₂.

In other examples, L¹ can be selected from any one of the following linker groups, in which it will be appreciated that either end of the linker group can be joined to either the A ring or B ring:

| | | |
|---|---|---|
| C₂alkyl/alkenyl | C₃alkyl/alkenyl | C₄alkyl/alkenyl |
| —CH₂—CH₂— | —CH₂—CH₂—CH₂— | —CH₂—CH₂—CH₂—CH₂— |
| —CH═CH— (E/Z) | —CH═CH—CH₂— (E/Z) | —CH═CH—CH₂—CH₂— (E/Z) |
| —O—CH₂— | —O—CH₂—CH₂— | —CH₂—CH═CH—CH₂— (E/Z) |
| —S—CH₂— | —CH₂—O—CH₂— | —CH═CH—CH═CH— (E/Z) |
| —NH—CH₂— | —O—CH₂—O— | —O—CH₂—CH₂—CH₂— |
| —NH—C(═O)— | —S—CH₂—CH₂— | —CH₂—O—CH₂—O— |
| —N(CH₃)—CH₂— | —CH₂—S—CH₂— | —S—CH₂—CH₂—CH₂— |
| —N(Et)—CH₂— | —S—CH₂—O— | —NH—C(═O)—CH₂—CH₂— |
| —O—C(═O)— | —NH—CH₂—CH₂— | —CH₂—NH—C(═O)—CH₂— |
| —C(═O)—CH₂— | —NH—C(═O)—CH₂— | —CH₂—CH₂—CH₂—CH₂— |

In other examples, each of the L¹ groups described above may be further substituted with one or more groups selected from halo and OH.

The “one or more” groups as described herein for optional substitution may be 1 to 6 groups, 1 to 5 groups, 1 to 4 groups, 1 to 3 groups, 1 or 2 groups, or 1 group.

The “one or more” groups as described herein for optional interruption may be 1 to 6 groups, 1 to 5 groups, 1 to 4 groups, 1 to 3 groups, 1 or 2 groups, or 1 group.

### B-Ring Groups

In Formula 1, the B ring groups can comprise X¹, X², X³, X⁴, and X⁵.

In one example, X¹, X², X³, X⁴, and X⁵, can each be independently selected from hydrogen, halo, OH, C₁₋₆alkyl, C₁₋₆alkylhalo, OC₁₋₆alkyl, OC₁₋₆alkylhalo, C(═O)OH, C(=O)O(C₁₋₁₀alkyl), and NO₂. Any two X groups can also join together to form an aryl group, for example X² and X³ can join together to form an aryl group (e.g. phenyl or naphthyl), such that the B ring is a substituted or unsubstituted naphthyl or anthracenyl group. In another example, X¹ and X² join together to form an aryl group and X⁴ and X⁵ join together to form an aryl group (e.g. phenyl group), such that the B ring is a substituted or unsubstituted anthracenyl group. The aryl group can be unsubstituted or substituted, for example substituted with one or more groups selected from halo, OH, C₁₋₆alkyl, C₁₋₆alkylhalo, OC₁₋₆alkyl, OC₁₋₆alkylhalo, C(═O)OH, C(=O)O(C₁₋₁₀alkyl), and NO₂. The aryl group can be a monocyclic (e.g. phenyl) or bicyclic group (e.g. naphthyl or linked biphenyl group).

In another example, X¹, X², X³, X⁴, and X⁵, can each be independently selected from hydrogen, halo, OH, C₁₋₆alkyl, C₁₋₆alkylhalo, OC₁₋₆alkyl, OC₁₋₆alkylhalo, and X² and X³ can join together to form a phenyl group.

The phenyl group can be unsubstituted or substituted, for example substituted with one or more groups selected from halo, OH, C₁₋₆alkyl, C₁₋₆alkylhalo, OC₁₋₆alkyl, OC₁₋₆alkylhalo, C(═O)OH, C(=O)O(C₁₋₁₀alkyl), and NO₂.

In another example, X¹, X², X³, X⁴, and X⁵, can each be independently selected from hydrogen, halo, OH, C₁₋₆alkyl, C₁₋₆alkylhalo, OC₁₋₆alkyl, OC₁₋₆alkylhalo.

In another example, X¹, X⁴, and X⁵, can each be independently selected from hydrogen, halo, OH, C₁₋₆alkyl, C₁₋₆alkylhalo, OC₁₋₆alkyl, OC₁₋₆alkylhalo, and X² and X³ are joined together to form a phenyl group. The phenyl group can be unsubstituted or substituted, for example substituted with one or more groups selected from halo, OH, C₁₋₆alkyl, C₁₋₆alkylhalo, OC₁₋₆alkyl, OC₁₋₆alkylhalo, C(═O)OH, C(=O)O(C₁₋₁₀alkyl), and NO₂.

In another example, a compound of Formula 1 can be provided by a compound of Formula 1a as follows:

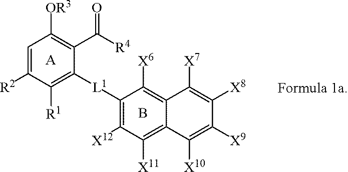

For Formula 1a, X⁶, X⁷, X⁸, X⁹, X¹⁰, X¹¹, and X¹², can each be independently selected from hydrogen, halo, OH, C₁₋₆alkyl, C₁₋₆alkylhalo, OC₁₋₆alkyl, OC₁₋₆alkylhalo, C(═O)OH, C(=O)O(C₁₋₁₀alkyl), and NO₂.

Each of the above various embodiments or examples of R¹, R², R³, R⁴, and L¹, may also apply to provide various independently selected further embodiments or examples of compounds of Formula 2.

### Example Compounds of Formula 1

Example compounds of Formula 1 can be selected from any one of the compounds in the following Table 1.

TABLE 1

Example Compounds

| | | |
|---|---|---|
| Chemical Structure | Chemical Name | Ref. |
| 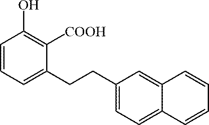 [SMILES: O=C(O)c1c(O)cccc1CCc1ccc2ccccc2c1] | 2-hydroxy-6-(2-(naphthalen-2-yl)ethyl)benzoic acid | GB001 (SM4) |
| 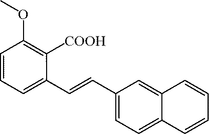 [SMILES: COc1cccc(/C=C/c2ccc3ccccc3c2)c1C(=O)O] | (E)-2-methoxy-6-(2-(naphthalen-2-yl)vinyl)benzoic acid | GB002 |
| 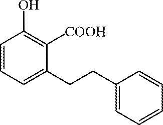 [SMILES: O=C(O)c1c(O)cccc1CCc1ccccc1] | 2-hydroxy-6-phenethylbenzoic acid | GB003 |
| 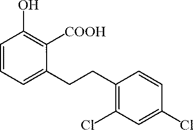 [SMILES: O=C(O)c1c(O)cccc1CCc1ccc(Cl)cc1Cl] | 2-(2,4-dichlorophenethyl)-6-hydroxybenzoic acid | GB004 |
| 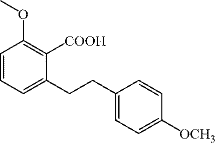 [SMILES: COc1ccc(CCc2cccc(OC)c2C(=O)O)cc1] | 2-methoxy-6-(4-methoxyphenethyl)benzoic acid | GB005 |
| 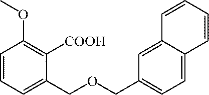 [SMILES: COc1cccc(COCc2ccc3ccccc3c2)c1C(=O)O] | 2-methoxy-6-((naphthalen-2-ylmethoxy)methyl)benzoic acid | GB006 |
| 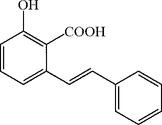 [SMILES: O=C(O)c1c(O)cccc1/C=C/c1ccccc1] | (E)-2-hydroxy-6-styrylbenzoic acid | GB007 |
| 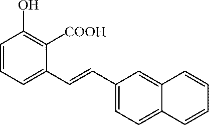 [SMILES: O=C(O)c1c(O)cccc1/C=C/c1ccc2ccccc2c1] | (E)-2-hydroxy-6-(2-(naphthalen-2-yl)vinyl)benzoic acid | GB008 |
| 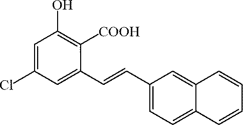 [SMILES: O=C(O)c1c(O)cc(Cl)cc1/C=C/c1ccc2ccccc2c1] | (E)-4-chloro-2-hydroxy-6-(2-(naphthalen-2-yl)vinyl)benzoic acid | GB009 |
| 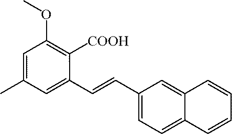 [SMILES: COc1cc(C)cc(/C=C/c2ccc3ccccc3c2)c1C(=O)O] | (E)-2-methoxy-4-methyl-6-(2-(naphthalen-2-yl)vinyl)benzoic acid | GB010 |
| 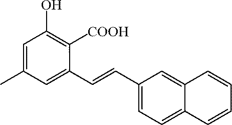 [SMILES: Cc1cc(O)c(C(=O)O)c(/C=C/c2ccc3ccccc3c2)c1] | (E)-2-hydroxy-4-methyl-6-(2-(naphthalen-2-yl)vinyl)benzoic acid | GB011 |
| 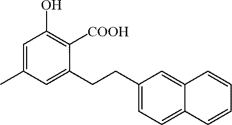 [SMILES: Cc1cc(O)c(C(=O)O)c(CCc2ccc3ccccc3c2)c1] | 2-hydroxy-4-methyl-6-(2-(naphthalen-2-yl)ethyl)benzoic acid | GB012 |
| 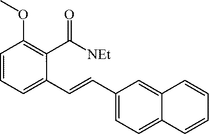 | (E)-N,N-diethyl-2-methoxy-6-(2-(naphthalen-2-yl)vinyl)benzamide | GB013 |
| 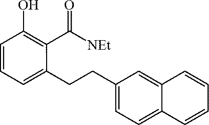 | N,N-diethyl-2-hydroxy-6-(2-(naphthalen-2-yl)ethyl)benzamide | GB014 |
| 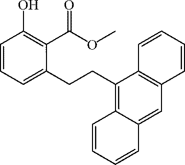 [SMILES: COC(=O)c1c(O)cccc1CCc1c2ccccc2cc2ccccc12] | methyl 2-(2-(anthracen-9-yl)ethyl)-6-hydroxybenzoate | GB015 |
| 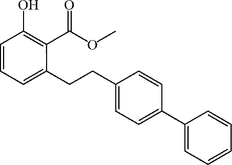 [SMILES: COC(=O)c1c(O)cccc1CCc1ccc(-c2ccccc2)cc1] | methyl 2-(2-([1,1′-biphenyl]-4-yl)ethyl)-6-hydroxybenzoate | GB016 |
| 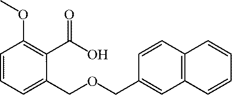 [SMILES: COc1cccc(COCc2ccc3ccccc3c2)c1C(=O)O] | 2-methoxy-6-((naphthalen-2-ylmethoxy)methyl)benzoic acid | GB017 |
| 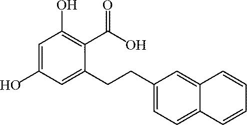 [SMILES: O=C(O)c1c(O)cc(O)cc1CCc1ccc2ccccc2c1] | 2,4-dihydroxy-6-(2-(naphthalen-2-yl)ethyl)benzoic acid | GB018 |
| 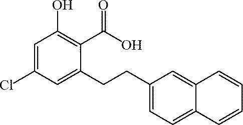 [SMILES: O=C(O)c1c(O)cc(Cl)cc1CCc1ccc2ccccc2c1] | 4-chloro-2-hydroxy-6-(2-(naphthalen-2-yl)ethyl)benzoic acid | GB019 |
| 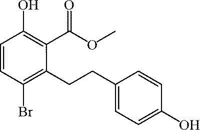 [SMILES: COC(=O)c1c(O)ccc(Br)c1CCc1ccc(O)cc1] | methyl 3-bromo-6-hydroxy-2-(4-hydroxyphenethyl)benzoate | GB020 |
| 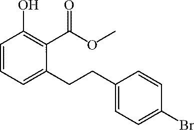 [SMILES: COC(=O)c1c(O)cccc1CCc1ccc(Br)cc1] | methyl 2-(4-bromophenethyl)-6-hydroxybenzoate | GB021 |
| 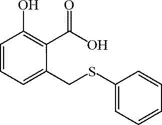 [SMILES: O=C(O)c1c(O)cccc1CSc1ccccc1] | 2-hydroxy-6-((phenylthio)methyl)benzoic acid | GB022 |
| 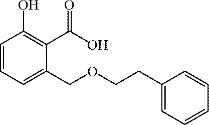 [SMILES: O=C(O)c1c(O)cccc1COCCc1ccccc1] | 2-hydroxy-6-(phenethoxymethyl)benzoic acid | GB023 |
| 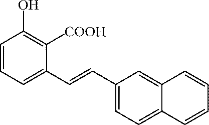 [SMILES: O=C(O)c1c(O)cccc1/C=C/c1ccc2ccccc2c1] | (E)-2-hydroxy-6-(2-(naphthalen-2-yl)vinyl)benzoic acid | GB024 |
| 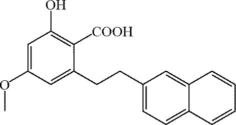 [SMILES: COc1cc(O)c(C(=O)O)c(CCc2ccc3ccccc3c2)c1] | 2-hydroxy-4-methoxy-6-(2-(naphthalen-2-yl)ethyl)benzoic acid | GB025 |
| 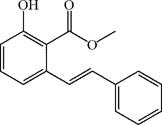 [SMILES: COC(=O)c1c(O)cccc1/C=C/c1ccccc1] | methyl (E)-2-hydroxy-6-styrylbenzoate | GB026 |
| 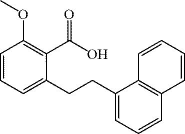 [SMILES: COc1cccc(CCc2cccc3ccccc23)c1C(=O)O] | 2-methoxy-6-(2-(naphthalen-1-yl)ethyl)benzoic acid | GB027 |
| 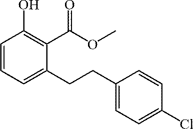 [SMILES: COC(=O)c1c(O)cccc1CCc1ccc(Cl)cc1] | methyl 2-(4-chlorophenethyl)-6-hydroxybenzoate | GB028 |

The compounds of the present invention also include stereoisomers of the compounds described herein and compositions comprising more than one compound of the invention may, where applicable, include such stereoisomers, for example E/Z isomers, either individually or admixed in any proportions. Stereoisomers may include, but are not limited to, enantiomers, diastereomers, racemic mixtures, and combinations thereof. Such stereoisomers can be prepared and separated using conventional techniques, either by reacting enantiomeric starting materials, or by separating isomers of compounds and prodrugs of the present invention. Isomers may include geometric isomers. Examples of geometric isomers include, but are not limited to, trans isomers or cis isomers (E/Z) across a double bond. Other isomers are contemplated among the compounds of the present invention. The isomers may be used either in pure form or in admixture with other isomers of the compounds described herein.

The compounds may optionally be provided in a composition that is enantiomerically or diastereomercially enriched, such as a mixture of enantiomers or diastereomers in which one enantiomer or diastereomer is present in excess, in particular, to the extent of 95% or more, 96% or more, 97% or more, 98% or more, or 99% or more, including 100%.

The compounds may be utilized per se or in the form of a pharmaceutically acceptable ester, amide, salt, solvate, prodrug, or isomer, as appropriate. For example, the compound may be provided as a pharmaceutically acceptable salt. If used, a salt of the drug compound should be both pharmacologically and pharmaceutically acceptable, but non-pharmaceutically acceptable salts may be used in the preparation of the compounds. Such pharmacologically and pharmaceutically acceptable salts can be prepared by reaction of the drug with an organic or inorganic acid, using standard methods detailed in the literature. Examples of pharmaceutically acceptable salts or solvates have been previously described.

### Preparation of Compounds of Formula 1

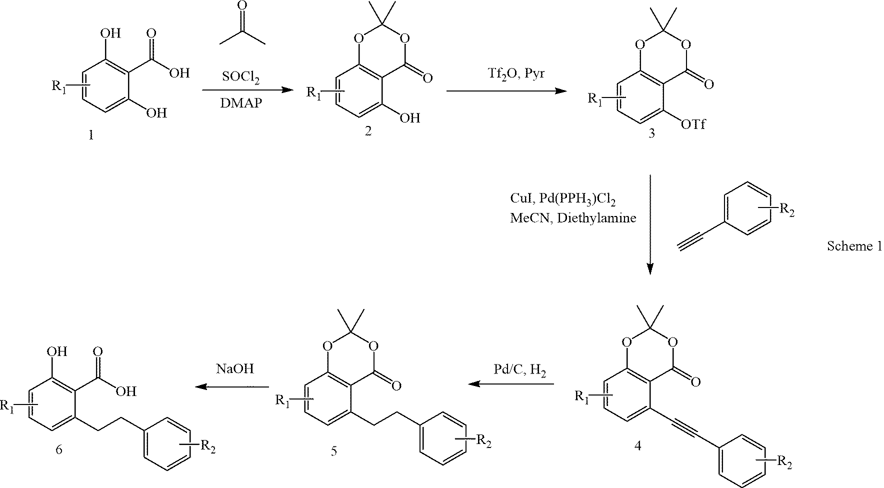

The compounds of Formula 1 may be prepared generally according to Scheme 1 above. For example, a salicylic acid derivative compound 1 can provide an A ring scaffold and be protected to form a cyclic lactam compound 2. A solution of compound 1, acetone and DMAP in DME (30 mL) can be provided and SOCl₂ added at about 0° C.). The mixture can be stirred at 0° C. for about an hour and then stirred at room temperature. The resulting mixture can be quenched with water and purified to afford compound 2.

The free hydroxyl group of compound 2 can be activated by forming a triflate. A solution of compound 2 and pyridine in DCM (50 mL) along with Tf₂O can be stirred at about 0° C. for about an hour. The mixture can be purified to provide compound 3.

The activated compound 3 can then be reacted with a linker-B ring scaffold. For example, a mixture of compound 3, 2-ethynylnaphthalene, Pd(PPh₃)₂Cl₂, diethylaminein and Cul in MeCN can be heated to reflux for about 2 hours under an atmosphere of N₂. The reaction mixture can be purified to provide a compound 4, which is a protected lactam derivative compound of Formula 1 comprising an unsaturated linker group.

Compound 4 can be modified to reduce the unsaturated linker group to a fully saturated alkyl linker group. For example, a mixture of compound 4 and Pd/C (10% on activated carbon, 500 mg) in MeOH can be stirred at room temperature for about 2 hours under an atmosphere of H₂ (1 atm). The mixture can be worked up and purified to provide a compound 5.

Compound 5 can then be deprotected to provide a compound of Formula 1. A mixture of compound 5, NaOH in THF and H₂O can be heated at 80° C. for about 16 hours. The mixture can be acidified with 1N HCl to pH 2-3 and extracted with EtOAc. The combined organic phases can be concentrated and purified to provide a compound 6.

It will be appreciated that the compounds may be prepared according to other synthetic approaches, and the above scheme provides one example of a synthetic approach.

### Pharmaceutical Compositions

In another aspect, there is provided a pharmaceutical composition comprising a compound of Formula 1, or any salt, stereoisomer, or solvate thereof according to any aspect, embodiment or example thereof as described herein, and a pharmaceutically acceptable carrier, diluent and/or excipient.

Suitably, the pharmaceutically acceptable carrier, diluent and/or excipient may be or include one or more of diluents, solvents, pH buffers, binders, fillers, emulsifiers, disintegrants, polymers, lubricants, oils, fats, waxes, coatings, viscosity-modifying agents, glidants and the like.

Diluents may include one or more of microcrystalline cellulose, lactose, mannitol, calcium phosphate, calcium sulfate, kaolin, dry starch, powdered sugar, and the like. Binders may include one or more of povidone, starch, stearic acid, gums, hydroxypropylmethyl cellulose and the like. Disintegrants may include one or more of starch, croscarmellose sodium, crospovidone, sodium starch glycolate and the like. Solvents may include one or more of ethanol, methanol, isopropanol, chloroform, acetone, methylethyl ketone, methylene chloride, water and the like. Lubricants may include one or more of magnesium stearate, zinc stearate, calcium stearate, stearic acid, sodium stearyl fumarate, hydrogenated vegetable oil, glyceryl behenate and the like. A glidant may be one or more of colloidal silicon dioxide, talc or cornstarch and the like. Buffers may include phosphate buffers, borate buffers and carbonate buffers, although without limitation thereto. Fillers may include one or more gels inclusive of gelatin, starch and synthetic polymer gels, although without limitation thereto. Coatings may comprise one or more of film formers, solvents, plasticizers and the like. Suitable film formers may be one or more of hydroxypropyl methyl cellulose, methyl hydroxyethyl cellulose, ethyl cellulose, hydroxypropyl cellulose, povidone, sodium carboxymethyl cellulose, polyethylene glycol, acrylates and the like. Suitable solvents may be one or more of water, ethanol, methanol, isopropanol, chloroform, acetone, methylethyl ketone, methylene chloride and the like. Plasticizers may be one or more of propylene glycol, castor oil, glycerin, polyethylene glycol, polysorbates, and the like.

Reference is made to the Handbook of Excipients 6^{th} Edition, Eds. Rowe, Sheskey & Quinn (Pharmaceutical Press), which provides non-limiting examples of excipients which may be useful according to the present disclosure. Other pharmaceutical excipients and/or additives suitable for use in the compositions according to the present disclosure are listed in “Remington: The Science & Practice of Pharmacy”, 19.sup.th ed., Williams & Williams, (1995), and in the “Physician’s Desk Reference”, 52.sup.nd ed., Medical Economics, Montvale, N.J. (1998), and in “Handbook of Pharmaceutical Excipients”, Third Ed., Ed. A. H. Kibbe, Pharmaceutical Press, 2000.

It will be appreciated that the choice of pharmaceutically acceptable carriers, diluents and/or excipients will, at least in part, be dependent upon the mode of administration of the formulation. By way of example only, the composition may be in the form of a tablet, capsule, caplet, powder, an injectable liquid, a suppository, a slow release formulation, an osmotic pump formulation or any other form that is effective and safe for administration.

### Dosages

The amount of active ingredient that is required to achieve a therapeutic effect will, of course, vary with the particular compound, the route of administration, the subject under treatment, including the type, species, age, weight, sex, and medical condition of the subject being treated, and the renal and hepatic function of the subject, and the particular condition, disorder or disease being treated, as well as its severity. An ordinary skilled physician or clinician can readily determine and prescribe the effective amount of the drug required to prevent or treat the condition, disorder or disease.

Dosages of a compound of Formula 1, or salt, solvate or stereoisomer thereof, when used for the indicated effects, may range between, for example, about 0.01 mg per kg of body weight per day (mg/kg/day) to about 1000 mg/kg/day. In one example, the dosage of a compound of Formula 1, or salt, solvate or stereoisomer thereof, is between about 0.01 and 1000, 0.1 and 500, 0.1 and 100, 1 and 50 mg/kg/day. In one example, the dosage of a compound of Formula 1, or salt, solvate or stereoisomer thereof, is between about 0.01 and 1000 mg/kg/day. In one example, the dosage of a compound of Formula 1, or salt, solvate or stereoisomer thereof, is between about 0.1 and 100 mg/kg/day. In one example, the dosage of a compound of Formula 1, or salt, solvate or stereoisomer thereof, is greater than about 0.01, 0.1, 1, 10, 20, 50, 75, 100, 500, 1000 mg/kg/day. In one example, the dosage of a compound of Formula 1, or salt, solvate or stereoisomer thereof, is less than about 5000, 1000, 75, 50, 20, 10, 1, 0.1 mg/kg/day.

A compound of Formula 1, or salt, solvate or stereoisomer thereof, may for example be administered as a single daily dose, or otherwise the total daily dosage may be administered in divided doses of two, three, or four times daily. In one example, the compound of Formula 1, or salt, solvate or stereoisomer thereof, may be dosed less frequently than once per day, for example once per two days, three days, four days, five days, six days, or once per week.

### Indications/Applications

The disclosure also provides a use of a compound of Formula 1 as defined according to any aspects, embodiments or examples as described herein, as an antiviral agent or for treating a viral disease or condition or a viral associated disease or condition.

Accordingly, the present disclosure provides a method of treating a viral disease or condition by administration of an antiviral compound to a subject in need of treatment thereof, wherein the antiviral compound is a compound of Formula 1, or pharmaceutically acceptable salt, solvate, or stereoisomer thereof, according to any aspects, embodiments or examples thereof as described herein.

In one example, the compounds described herein are particularly useful for treating or preventing a SOX18-dependent viral disease or condition. A “SOX18-dependent viral disease or condition” is referred to herein as one which involves SOX18 activity. In one example, the SOX18 activity includes contacting and/or binding of SOX18 to a DNA sequence and/or a protein. In a further example, the protein is selected form the group consisting of SOX7, RBPJ, XRCC5, SOX18, ILF3, DDX17 and any combination of thereof.

In one example, the compound of Formula 1 described herein inhibit, prevent or reduce the SOX18 activity in a subject. In another example, the compound of Formula 1 demonstrates one or more of the following activities as determined in cell culture such as KLEC or luciferase assay as described herein. In one example, the compound of Formula 1 described herein selectively inhibit SOX18 activity.

The SOX18-dependent viral disease or condition may be selected from the group consisting of Kaposi sarcoma, AIDS-related lymphoproliferative disorder, angio-immunoblastic T-cell lymphoma, Burkitt’s lymphoma, Hodgkin’s lymphoma, Non-Hodgkin’s lymphoma, Leiomyosarcoma, breast cancer, Gastric carcinoma, Nasal T/NK cell lymphoma, T/NK cell lymphoma, nasopharyngeal carcinoma, CMV retinitis of the eyes, pneumonia, gastrointestinal ulcer, oral herpes, genital herpes and joint inflammation.

An anti-viral compound of the disclosure can be combined with a further agent such as an antiretroviral, retinoid or chemotherapeutic agent. The anti-viral agent may also be combined with a treatment such as radiation therapy, photodynamic treatment or cryosurgery.

Examples of agents that may be used in combination with a compound of the disclosure include antivirals such as ganciclovir or forcarnet; retinoids such as alitretinoin and chemotherapy agents such asdoxorubicin, daunorubicin, paclitaxel, vinorelbine, bleomycin, and etoposide.

The disclosure also provides use of a compound of Formula 1 as defined herein in the manufacture of a medicament for treating a viral disease or condition or a viral associated disease or condition.

In another example, the antiviral compound of Formula 1 inhibits SOX18-SOX18 homodimerisation. In another example, the antiviral compound of Formula 1 inhibits SOX18-RBPJ heterodimersation.

The present disclosure also provides a method for inhibiting replication of a herpesvirus and/or treating a viral disease or condition caused by a herpesvirus in a subject comprising administering to the subject a compound of Formula 1 as described herein. In one example, the viral disease or condition is SOX18 –dependent.

The herpesvirus may be selected from the group consisting of Alpha Herpesviruses, Beta Herpesviruses, Gamma₁ Herpesviruses and Gamma₂ Herpesviruses. In one example, the herpesevirus is selected from the group consisting of Kaposi sarcoma herpesvirus (KSHV), Epstein-Barr virus (EBV), cytomegalovirus (CMV), Vesicular stomatitis virus (VSV), rhesus lymocryptovirus (rLCV), Herpes simplex virus 1 (HSV-1), Herpes simplex virus (HSV-2) and Ross river virus (RRV).

In another example, the viral disease or condition is selected from any one of the following or from the group consisting of:
(i) Kaposi sarcoma caused by KSHV;
(ii) AIDS-related lymphoproliferative disorder caused by KSHV;
(iii) Angio-immunoblastic T-cell lymphoma caused by EBV;
(iv) Burkitt’s lymphoma caused by EBV;
(v) Hodgkin’s lymphoma caused by EBV;
(vi) Non-Hodgkin’s lymphoma caused by EBV;
(vii) Leiomyosarcoma caused by EBV;
(viii) Breast cancer caused by EBV;
(ix) Gastric carcinoma caused by EBV
(x) Nasal T/NK cell lymphoma caused by EBV;
(xi) T/NK cell lymphoma caused by EBV;
(xii) Nasopharyngeal carcinoma caused by EBV
(xiii) CMV retinitis of the eyes caused by CMV;
(xiv) pneumonia caused by CMV;
(xv) gastrointestinal ulcers caused by CMV;
(xvi) a condition or symptom caused by VSV;
(xvii) Oral herpes caused by HSV-1;
(xviii) Genital herpes caused by HSV-2; and
(xix) joint inflammation or rash caused by RRV.

In another example, Kaposi sarcoma is selected from any one of or the group consisting of classic Kaposi sarcoma, endemic Kaposi sarcoma, AIDS-related Kaposi sarcoma, and iatrogenic Kaposi sarcoma.

### Kaposi Sarcoma

Kaposi sarcoma herpesvirus (KSHV) is one of nine species in the genus Rhadinovirus of the subfamily Gammaherpesvirus in the family Herpesviridae. KSHV, like other herpesviruses establishes lifelong infection in the infected hosts and maintains the viral genome as extra-chromosomal episomes in a latent state. The virus encodes a limited number of genes for persistence without being recognized by the host immune surveillance. Latency Associated Nuclear Antigen (LANA) is one of the proteins expressed in all latently infected cells (Rainbow L, et al. (1997) J Virol 71: 5915-5921; Renne R, et al., (1998) J Virol 72: 5182-5188).

LANA is considered an oncogenic protein because of its role in modulating cellular pathways required to induce/promote tumorigenesis (Moore PS, Chang Y (1998) J Natl Cancer Inst Monogr. pp 65-71). Along with its role in modulation of various cellular and viral pathways, LANA is critical for maintaining the viral genome in infected cells (Cotter MA, 2nd, Robertson ES (1999) Virology 264: 254-264; Ye FC, et al. (2004) J Virol 78: 11121-11129).

LANA docks onto the host chromatin through the amino terminal chromatin-binding domain (CBD) and tethers the viral genome to the host chromosome by binding to the DNA binding domain of the carboxyl terminus within the terminal repeats (Barbera AJ, et al. (2006) Science 311: 856-861; Cotter MA, 2nd, Subramanian C, Robertson ES (2001) Virology 291: 241-259).

The KSHV genome has multiple reiterated copies of the terminal repeats (TR), which are proposed to be the region required for circularisation of the genome. Each terminal repeat unit is a 801 bp long high GC content DNA element and was shown to contain the latent origin, or replication initiation site similar to EBV. Each TR unit has two LANA binding sites (a high affinity site LBS1 and a lower affinity site LBS2). A 31 bp long sequence upstream of the LANA binding sequence is mapped as a replicator element (RE) important for replication initiation. Each TR unit has a replicator element.

Work has demonstrated the presence of an additional replication site at the left end of the KSHV genome (Verma SC, et al., (2007) Cell Host Microbe 2:106-118). This replication site does not require expression of LANA in trans, and is referred to as an autonomous replication origin (oriA, as referred to herein). Accordingly, replication initiation events can occur throughout the KSHV genome, which is distinctly different from earlier conclusions that replication initiates from a specific site within the terminal repeats.

### Types of Kaposi Sarcoma

There are a number of different types of KS which are defined by the different populations in which the disease develops. Classic (or Mediterranean) KS occurs in elderly people of Mediterranean, Eastern European, and Middle Eastern heritage and occurs more commonly in men than in women. Patients typically have one or more lesions on the legs, ankles, or the soles of the feet. In comparison with other types of KS, the lesions in this type do not grow as quickly, and new lesions do not develop as often. People who get classic KS come from areas where KSHV infection is more common than in the US or Northern Europe. The immune system of people with classic KS is not as weakened as those who have epidemic KS (see below); however, old age may naturally weaken the immune system, thus making people more likely to develop KS if they already have a KSHV infection.

Endemic KS occurs in people living in Equatorial Africa and is sometimes called African KS. KSHV infection is much more common in Africa than in other parts of the world, increasing the chance of developing KS. There appear to be other factors in Africa that contribute to the development of KS since the disease affects a broader group of people that includes children and women. Endemic KS tends to occur in younger people (usually under age 40). In some parts of Africa, KS is currently considered the most common cancer (Horenstein et al., (2008); J. Cutan. Pathol. 35(Suppl. 2): 40-44).

The most common type of KS in the United States is epidemic or AIDS-related KS. This type of KS develops in individuals who are infected with HIV, the virus that causes AIDS. The severe immunosuppression caused by AIDS increases the likelihood of the development of KS in individuals already infected with KSHV. This more aggressive form of KS was first noted in young homosexual men in the 1970s. In addition to departing from the usual ethnic predisposition, the disease manifested with lesions that occurred at any site and that tended to spread more rapidly to the lymph nodes and visceral organs, such as the gastrointestinal and respiratory tracts. Gastrointestinal (GI) involvement is generally asymptomatic and does not affect prognosis, while lung involvement frequently is symptomatic and adversely affects prognosis. The disease progressed very rapidly and many patients died within one year, despite drug chemotherapy regimens. Treatment of HIV infection with highly active antiretroviral therapy (HAART) has decreased the incidence of epidemic KS and can often keep advanced KS from developing. The clinical course of AIDS-KS is variable, ranging from a very indolent process requiring little, if any therapy, to a rapidly progressive and fatal disease

When KS develops in people whose immune systems have been suppressed after an organ transplant it is called iatrogenic, or transplant-associated KS or immunosuppression-associated KS. Most transplant patients take immunosuppressant drugs, such as rapamycin, to prevent organ rejection. The immunosuppression caused by these drugs increases the likelihood that individuals infected with KSHV will develop KS. Stopping the immune suppressing drugs or lowering their dose often makes KS lesions disappear or get smaller.

### EXAMPLES

### Compound Preparations

Compound GB001 (SM4) was synthesised by the following process as shown in Scheme 2:

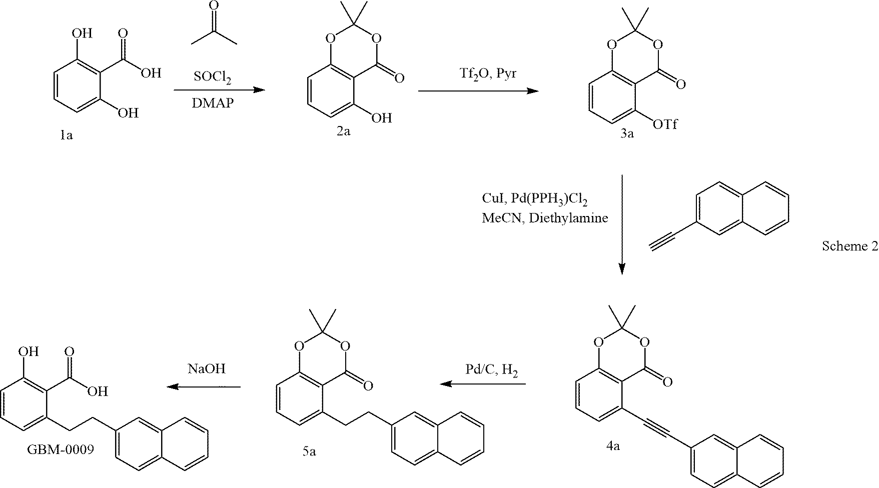

To a solution of compound 1a (51.9 mmol), acetone (67.53 mmol) and DMAP (2.59 mmol) in DME (30 mL) was added SOCl₂ dropwise at 0° C. The mixture was stirred at 0° C. for 1 hour then stirred at room temperature for 16 hours. The resulting mixture was quenched with water and purification by flash column chromatography on silica gel (0 to 100% EtOAc in PE) to afford compound 2a (62.6 %) as a yellow solid.

To a solution of compound 2a (25.8 mmol) and pyridine (92.7 mmol) in DCM (50 mL) was added Tf₂O (30.9 mmol) at 0° C. The mixture was stirred at room temperature for 1 hour. Solvent was removed under vacuum and the crude was purified by flash column chromatography on silica gel (eluting with 0 to 50% EtOAc in PE) to give compound 3a (65.1%) as a yellow solid.

A mixture of compound 3a (3.06 mmol), 2-ethynylnaphthalene (3.36 mmol), Pd(PPh₃)₂Cl₂ (0.055 Mmol), diethylaminein (6.12 mmol) and Cul (0.3 mmol) in MeCN (15 mL) was heated reflux for 2 hours under an atmosphere of N2. The reaction mixture was filtered through celite. The filtrate was concentrated under vacuum and purified by flash column chromatography on silica gel (0 to 50% EtOAc in PE) to give compound 4a (75.22%) as white solid.

A mixture of compound 4a (2.29 mmol) and Pd/C (10% on activated carbon, 500 mg) in MeOH (20 mL) was stirred at room temperature for 2 hours under an atmosphere of H₂ (1 atm). Then mixture was filtered through celite. The filtrate was concentrated and purified by flash column chromatography (0 to 50% EtOAc in PE) to give compound 5a (59.2%) as a white solid.

A mixture of compound 5a (1.36 mmol), NaOH (6.8 mmol) in THF (10 mL) and H₂O (10 mL) was heated at 80° C. for 16 hours. The mixture was acidified with 1 N HCl to pH 2-3 and extracted with EtOAc. The combined organic phases was concentrated and purified on a Biotage Isolera One (C18 column, eluting with 30% to 100% MeCN/H2O containing 0.1% HCOOH) to afford Compound 1 (GBM-0009, SM4) (88.16%) as a white solid.

Purity of Compound 1 has been assessed by HPLC-UV/MS, reporting a purity of 99.5% (by UV₂₅₄), and a correct [M-H] mass of 290.95, and ¹H-NMR with correct assignment and peak area for each proton.

The synthesis of GB003 was similar to that of GB001 by using a 3-ethynylphenylene instead of a 3-ethynylnaphthalene. The synthesis of GB002 and GB003 has been described previously generally above and in WO2018/112545, which its entire contents are herein incorporated by reference.

Compounds GB004, GB005 and GB006 were purchased from ABCr (Germany) and analyzed for purity by HPLC/MS.

### Inhibitor Treatments

The following inhibitors were used: GB001, GB002, GB003, GB004, GB005 and GB006.

KSHV-infected cells were incubated with the indicated inhibitor at the concentrations stated in the Figure panels for six days prior to analysis. HeLa cells were incubated with the indicated inhibitor at the concentrations stated in the Figure panels for 24 hours prior to analysis.

### KLEC Genome Copy Number Assay

Primary human dermal lymphatic (C-12216) endothelial cells (LECs) were purchased from Promocell and grown in Lonza EBM-2 (00190860) supplemented with EGMTM-2 MV Microvascular Endothelial SingleQuotsTM(CC-4147). Cell from passage 1-3 were used.

rKSHV.219 was produced from iSLK.219 cells reactivated using 0.2 µg/ml doxycycline and 1.35 mM NaB for 72 h. Supernatant was harvested, spun down (2000 rpm 5 min) and sterile filtered using 45 µm pore-size filters. Subsequently the supernatant was ultracentrifuged at 22000 rpm for 2 h. The concentrated virus was then aliquoted and stored at -80° C.

Virus titres were determined by infecting U2OS cells with serial dilutions of the concentrated virus preparation and assessing the amount of GFP+ or LANA+ cells 24 h post-infection by automated high-content microscopy.

Equal number of LECs were seeded into 6-well assay-plates and incubated at 37° C. & 5% CO2 until 80% confluency. After reaching confluency, LECs were infected with rKSHV.219 and incubated at 37° C. & 5% CO2 for 3 days, making sure that the cell density remained similar. Cell were subsequently mixed at a 2:3 ratio with uninfected LECs for 2 days. Once GFP expression and cell spindling was observed the culture medium was replaced with medium containing the experimental compounds with 0.25% DMSO (v/v) at concentrations from 0.1 uM to 50 uM. After 3 days of incubation, the cells were replenished with new medium containing experimental compounds and incubated for 3 more days.

After compound incubation, cells were collected and genomic and viral DNA was extracted using the NucleoSpin Tissue Kit (Macherey-Nagel, 740952) following the kit’s standard protocol. qPCR was subsequently completed (SYBR Green, ThermoFisher K0222) in triplicates using primers for viral K8.1 and Human genomic actin. K8.1 forward primer: AAAGCGTCCAGGCCACCACAGA (SEQ ID NO:1); reverse primer: GGCAGAAAATGGCACACGGTTAC (SEQ ID NO:2). Genomic acid forward primer: AGAAAATCTGGCACCACACC (SEQ ID NO:3); reverse primer: AACGGCAGAAGAGAGAACCA (SEQ ID NO:4).

### SOX18 Luciferase Reporter Assay

HeLa cells were cultured in DMEM containing 10% FCS, 1% L-glutamine and 1% pen/strep at 37° C. & 5% CO₂. Cells were seeded at a density of 7000 cells per well in 125 µL of the maintenance medium in a 96-well format for 24 hours.

Plasmids were transfected using FuGENE HD Transfection Reagent (Promega E2311) (1:4 DNA to reagent ratio) and Opti-MEM (ThermoFisher 31985062). OriA-luc negative control and experimental wells contained 25 ng OriA-luc plasmid and 10 ng SOX18 plasmid per well, positive control contained 25 ng OriA-luc plasmid and 10 ng mCherry plasmid per well. 7XTR-luc negative control and experimental wells contained 50 ng 7XTR-luc plasmid and 20 ng SOX18 plasmid per well, positive control contained 50 ng 7XTR-luc plasmid and 20 ng mCherry plasmid per well. Cell were incubated for 18 hours before cells were replenished with new medium containing experimental compounds and incubated for 24 hours.

Luminescence was measured by adding an equal volume of Steady-Glo reagent to cells at room temperature and luminescence was quantified using the FLUOstar plate reader (BMG Labtech, FLUOstar Omega; 4 sec intervals per well).

### Preparation of Compounds

A library of n-butanol fractions generated from a marine library collected across Australia and Antarctica was used for screening. Active fractions were fractionated into pure compounds re-assayed in the same way as original fractions.

A library of 2688 samples of marine invertebrate and alga collected across southern Australia and Antarctica was processed to generate an extract library suitable for high throughput bioassay. EtOH extracts were decanted, concentrated and partitioned into n-BuOH and H₂O phases, then transferred to deep 96-well plates, resulting in a >10-fold concentration of small molecules, while removing salts. The n-BuOH fraction (25 mg/mL w/v of dried residue) was used for screening, following 10- and 100-fold dilution (2.5 and 0.25 mg/mL). Active fractions were triturated with hexane, CH₂Cl₂ and MeOH, and fractionated into pure compounds by HPLC. All compounds were assayed in the same way as fractions.

Compound 1 (GB001, SM4) was purchased from EndoTherm GmbH (Germany) and analysed for purity by HP-LC/MS.

The synthesis of GB002 and GB004 has been described previously generally above and in WO2018/112545.

### Sox18 Activity of Compounds

Monkey kidney fibroblast-like cells (COS-7) were cultured at 37° C. and 5% CO₂ in DMEM (Life Technologies, 11995) with fetal bovine serum (FBS), sodium pyruvate, L-glutamine, penicillin, streptomycin, non-essential amino acids, and HEPES. Cells were grown in 96-well plates to 80% confluence and transfected with mouse plasmids pGL2 Vcam-1 promoter construct (VC1889) and pReceiver M49 SOX18, using X-tremeGENE HP DNA transfection reagent (Roche, 6366236001). After 4 hr of transfection, cells were incubated with compounds in 0.5% FBS medium for another 24 hr, before lysis and luciferase assay (Perkin Elmer, 6016711). Results are presented as %inhibition of the maximal signal observed in cells transfected with VCAM-1 and SOX18 without compound incubation.

### Statistical Analysis

Data was presented as the mean +/- SD of at least 3 independent experiments. An ordinary one-way ANOVA or two-way ANOVA was performed as appropriate followed by Dunnett’s post-hoc test for multiple comparisons.

### Example 1: Compounds and Their Activity on SOX18 Protein Interactions

The compounds examined in this study are described in Table 2 below.

TABLE 2

GBM compounds and functional activity

| Chemical Structure | Chemical Name | Ref. | %inhibition of Sox18 |
|---|---|---|---|
| 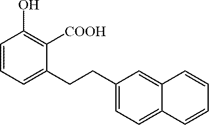 [SMILES: O=C(O)c1c(O)cccc1CCc1ccc2ccccc2c1] | 2-hydroxy-6-[2-(naphthalen-2-yl)ethyl]benzoic acid | GB001 | 99.10 |
| 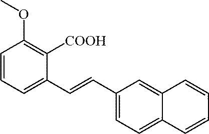 [SMILES: COc1cccc(/C=C/c2ccc3ccccc3c2)c1C(=O)O] | 2-methoxy-6-[(1E)-2-(naphthalen-2-yl)ethenyl]benzoic acid | GB002 | 30.78 |
| 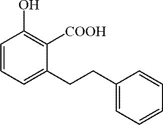 [SMILES: O=C(O)c1c(O)cccc1CCc1ccccc1] | 2-hydroxy-6-(2-phenylethyl)benzoic acid | GB003 | 49.18 |
| 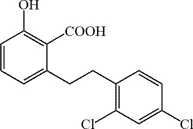 [SMILES: O=C(O)c1c(O)cccc1CCc1ccc(Cl)cc1Cl] | 2-(2,4-dichlorophenethyl)-6-hydroxybenzoic acid | GB004 | 48.26 |
| 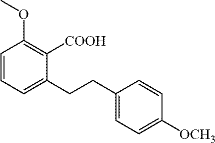 [SMILES: COc1ccc(CCc2cccc(OC)c2C(=O)O)cc1] | 2-methoxy-6-(4-methoxyphenethyl)benzoic acid | GB005 | 25.32 |
| 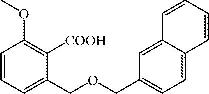 [SMILES: COc1cccc(COCc2ccc3ccccc3c2)c1C(=O)O] | 2-methoxy-6-((naphthalen-2-ylmethoxy)methyl)benzoic acid | GB006 | 19.33 |
| 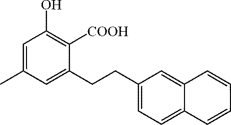 [SMILES: Cc1cc(O)c(C(=O)O)c(CCc2ccc3ccccc3c2)c1] | 2-hydroxy-4-methyl-6-(2-(naphthalen-2-yl)ethyl)benzoic acid | GB012 | 96.7 |
| 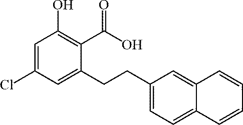 [SMILES: O=C(O)c1c(O)cc(Cl)cc1CCc1ccc2ccccc2c1] | 4-chloro-2-hydroxy-6-(2-(naphthalen-2-yl)ethyl)benzoic acid | GB019 | 54.02 |
| 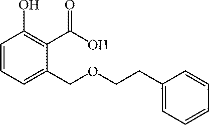 [SMILES: O=C(O)c1c(O)cccc1COCCc1ccccc1] | 2-hydroxy-6-(phenethoxymethyl)benzoic acid | GB023 | 78.07 |
| 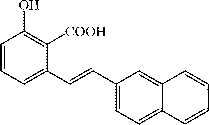 [SMILES: O=C(O)c1c(O)cccc1/C=C/c1ccc2ccccc2c1] | (E)-2-hydroxy-6-(2-(naphthalen-2-yl)vinyl)benzoic acid | GB024 | 100.21 |
| 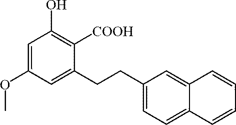 [SMILES: COc1cc(O)c(C(=O)O)c(CCc2ccc3ccccc3c2)c1] | 2-hydroxy-4-methoxy-6-(2-(naphthalen-2-yl)ethyl)benzoic acid | GB025 | 67.19 |
| 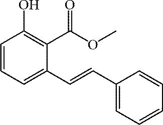 [SMILES: COC(=O)c1c(O)cccc1/C=C/c1ccccc1] | methyl (E)-2-hydroxy-6-styrylbenzoate | GB026 | 49.66 |
| 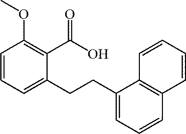 [SMILES: COc1cccc(CCc2cccc3ccccc23)c1C(=O)O] | 2-methoxy-6-(2-(naphthalen-1-yl)ethyl)benzoic acid | GB027 | 47.43 |
| 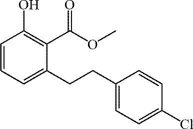 [SMILES: COC(=O)c1c(O)cccc1CCc1ccc(Cl)cc1] | methyl 2-(4-chlorophenethyl)-6-hydroxybenzoate | GB028 | 48.31 |

### Example 2: Involvement of 7XTR and OriA in Kaposi Sarcoma

Replication of latent Kaposi Sarcoma (KS) herpesevirus (KSHV), the virus responsible for Kaposi Sarcoma involves the terminal repeat region (TR) and to a minor extent, the OriA region of the KSHV genome.

Accordingly, the inventors used a luciferase assay to assess the role of SOX18 in the expression of the TR and OriA regions of the virus.

Activation of these genes was measured using HeLa cells that were transfected with luciferase-reporters harbouring either seven copies of the TR (7XTR) or the OriA promoter fused to OriLyt upstream of an SV40 promoter and a firefly luciferase reporter. ORF, that binds to the OriLyt and is a potent activator of this reporter (Chen J et al., (2009) Virology 386:290-302) was used as a positive control. In the presence of LANA, SOX18 expression increased the activity of the 7XTR reporter in a dose-dependent manner. SOX18 expression also increased the activity of the OriA+OriLyt reporter in an ORF50-independent manner. SOX18 did not change the activity of a reporter plasmid harbouring the ORF50 promoter (data not shown) supporting the specificity of the activation observed in the 7XTR and OriA+OriLyt reporters.

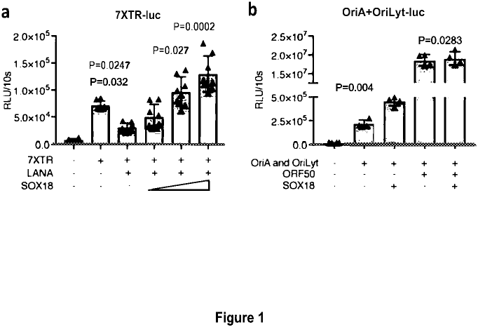

FIG. 1 shows that HeLa cells co-transfected with SOX18 demonstrate increased activity of 7XTR and OriA. This indicates that the activation of 7XTR and OriA are under control of SOX18. Data obtained from Gramolelli et al. 2020 Cancer Res.

Accordingly, the luciferase assay can be used as a read-out as to whether the compounds described herein are capable of interfering with SOX18 and hence replication of Kaposi Sarcoma.

### Example 3 SOX18 Activation of Gene Expression

The ability of the compounds to interference with SOX18 activation of gene expression was measured by two different assays.

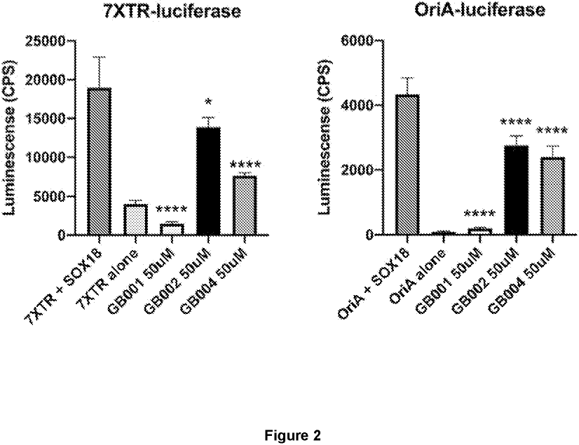

The first assay examined the ability of compound GB001 to inhibit SOX18 activity of the TR and OriA regions of the Kaposi sarcoma virus. FIG. 2 shows the results of the luciferase assay treatment of compounds GB001, GB002 and GB004 on HeLa cells transfected with 7xTR and OriA.

These results demonstrate that GB001, GB002 and GB004 interferes with SOX18-mediates gene transcription in Kaposi sarcoma.

The second assay measured the interaction between SOX18 and the VCAM-1 promoter (Hosking et al., (2004) J Biol. Chem. 297:5314-5322). Inhibition of this interaction demonstrates cell-based efficacy of the ability of a compound to modulate SOX18-mediated gene transcription. Without wishing to be bound by theory, it is understood that viral infection can induce an inflammatory response of endothelial cells, the latter being a result of VCAM-1 gene expression which is regulated by SOX18 activity (Huber J. (1994) J Virol. 68(6):3453-8). Put another way, the expression of VCAM-1 is driven by SOX18 so upon virus infection, SOX18 binds to the VCAM promoter and induces expression of VCAM.

The compounds described in Table 2 were tested by the VCAM-1 luciferase reporter assay. The results are shown in Table 2 under the column %inhibition of SOX18.

The compounds that showed the greatest level of inhibition were GB001 (99.10% inhibition), GB024 (100% inhibition), GB023 (78% inhibition) and GB012 (96.7% inhibition). As discussed above, the VCAM-1 assay may be suggestive of viral infection (Ou R et al., (2008) J Virol. 82(6):2952-2965; Pati S et al., (2001) 75(10):8660-73). Thus, the compounds were examined for their ability to inhibit VCAM-1 expression as a surrogate for their potential activity as viral inhibitors.

### Example 4 Effect of the Compounds on Viral Genome Copy Number

A KLEC assay was used to examine the ability of the compounds to inhibit viral copy number in virus infected primary human dermal lymphatic endothelial cells (LECs).

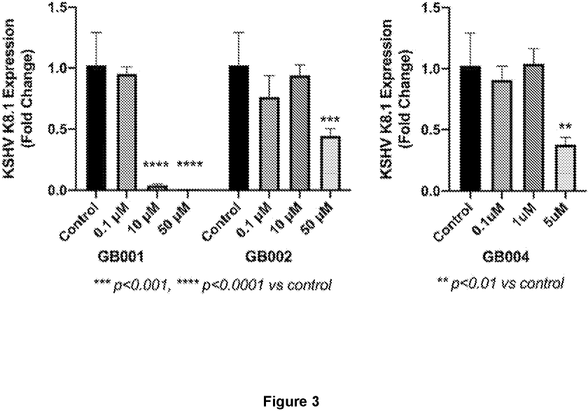

FIG. 3 shows the results for LECs infected with Kaposi sarcoma herpesvirus (KSHV). The results for three representative compounds (GB001, GB002 and GB004) is shown. Significant reduction in genome copy number as demonstrated by fold change was seen for GB001 at 10 µM and 50 µM; GB002 at 50 µM and GB004 at 5 µM.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method of treating a viral disease or condition in a subject comprising administering an antiviral compound to a subject in need of treatment thereof, wherein the antiviral compound is a compound of Formula 1: 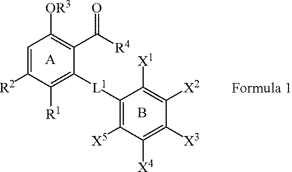
wherein
R¹ and R² are each independently selected from hydrogen, halo, OH, C₁₋₁₀alkyl, C₁₋₁₀alkylhalo, OC₁₋₁₀alkyl, OC₁₋₁₀alkylhalo, C₁₋₁₀alkenyl, C₁₋₁₀alkenylhalo, OC₁₋₁₀alkenyl, OC₁₋₁₀alkenylhalo;
R³ is selected from hydrogen, C₁₋₁₀alkyl, C₁₋₁₀alkylhalo, C₁₋₁₀alkenyl, C₁₋₁₀alkenylhalo;
R⁴ is selected from hydrogen, OH, C₁₋₁₀alkyl, OC₁₋₁₀alkyl, C₁₋₁₀alkylhalo, OC₁₋₁₀alkylhalo, NH₂, NH(C₁₋₁₀alkyl), and N(C₁₋₁₀alkyl)₂;
L¹ is selected from C₁₋₁₀alkyl, OC₁₋₁₀alkyl, C₁₋₁₀alkenyl, OC₁₋₁₀alkenyl, OC(═O), OC(=O)(C₁₋₁₀alkyl), NHC(═O), N(C₁₋₁₀alkyl)C(=O), OS(═O)₂, wherein each alkyl or alkenyl is uninterrupted or interrupted with one or more groups selected from O, OC(═O), NH, N(C₁₋₁₀alkyl), NHC(═O), S, and S(═O)₂, and unsubstituted or substituted with one or more groups selected from halo, OH, and C═O;
X¹, X², X³, X⁴, and X⁵, are each independently selected from hydrogen, halo, OH, C₁₋₁₀alkyl, C₁₋₁₀alkylhalo, OC₁₋₁₀alkyl, OC₁₋₁₀alkylhalo, C₁₋₁₀alkenyl, C₁₋₁₀alkenylhalo, OC₁₋₁₀alkenyl, OC₁₋₁₀alkenylhalo, C(═O)H, C(═O)OH, C(=O)O(C₁₋₁₀alkyl), and NO₂, and any two X groups can join together to form an aryl group unsubstituted or substituted with one or more groups selected from halo, OH, C₁₋₁₀alkyl, C₁₋₁₀alkylhalo, OC₁₋₁₀alkyl, OC₁₋₁₀alkylhalo, C₁₋₁₀alkenyl, C₁₋₁₀alkenylhalo, OC₁₋₁₀alkenyl, OC₁₋₁₀alkenylhalo, C(═O)H, C(═O)OH, C(=O)O(C₁₋₁₀alkyl), and NO₂;
or pharmaceutically acceptable salt, solvate, or stereoisomer thereof.

2. The method of claim 1, wherein R¹ and R² are hydrogen.

3. The method of claim 1, wherein R³ is hydrogen or C₁₋₆alkyl.

4. The method of claim 1, wherein R⁴ is OH or C₁₋₆alkyl.

5. The method of claim 1, wherein L¹ is selected from C₁₋₁₀alkyl or C₁₋₁₀alkenyl, wherein each alkyl or alkenyl is uninterrupted or interrupted with one or more groups selected from O, OC(═O), NH, N(C1-10alkyl), NHC(═O), S, and S(═O)₂; and unsubstituted or substituted with one or more groups selected from halo, OH, and C═O.

6. The method of claim 1, wherein L¹ is selected from C₂₋₆alkyl or C₂₋₆alkenyl, wherein each alkyl or alkenyl is uninterrupted or interrupted with one or more groups selected from O, NH, NHC(═O), S, and S(═O)₂; and unsubstituted or substituted with one or more groups selected from halo, OH, and C═O.

7. The method of claim 1, wherein L¹ is selected from C₂₋₄alkyl or C₂₋₄alkenyl, wherein each alkyl or alkenyl is uninterrupted or interrupted with one or more groups selected from O, NH, NHC(═O), S, and S(═O)₂.

8. The method of claim 1, wherein L¹ is selected from C₂₋₄alkyl or C₂₋₄alkenyl, wherein each alkyl or alkenyl is uninterrupted or interrupted with one or more groups selected from O and S.

9. The method of claim 1, wherein X¹, X², X³, X⁴, and X⁵, are each independently selected from hydrogen, halo, OH, C₁₋₆alkyl, C₁₋₆alkylhalo, OC₁₋₆alkyl, OC₁₋₆alkylhalo, C(═O)OH, C(=O)O(C₁₋₁₀alkyl), and NO₂; and X² and X³ can join together to form an aryl group unsubstituted or substituted with one or more groups selected from halo, OH, C₁₋₆alkyl, C₁₋₆alkylhalo, OC₁₋₆alkyl, OC₁₋₆alkylhalo, C(═O)OH, C(=O)O(C₁₋₁₀alkyl), and NO₂.

10. The method of claim 1, wherein X¹, X², X³, X⁴, and X⁵, are each independently selected from hydrogen, halo, OH, C₁₋₆alkyl, C₁₋₆alkylhalo, OC₁₋₆alkyl, OC₁₋₆alkylhalo, C(═O)OH, C(=O)O(C₁₋₁₀alkyl), and NO₂; and X² and X³ can join together to form a phenyl or naphthyl group unsubstituted or substituted with one or more groups selected from halo, OH, C₁₋₆alkyl, C₁₋₆alkylhalo, OC₁₋₆alkyl, OC₁₋₆alkylhalo, C(═O)OH, C(=O)O(C₁₋₁₀alkyl), and NO₂.

11. (canceled)

12. The method of claim 1, wherein the compound of Formula 1 is selected from any one of: 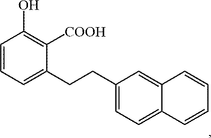 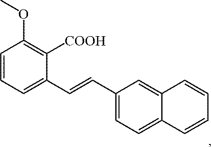 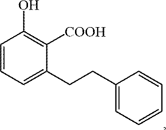 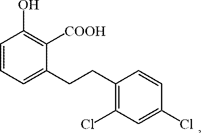 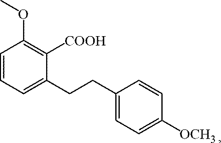 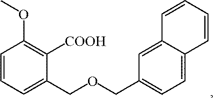 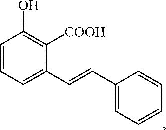 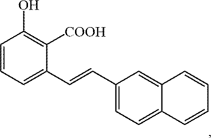 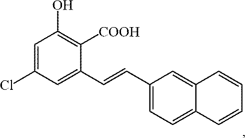 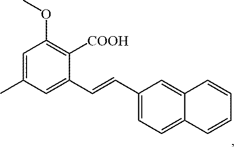 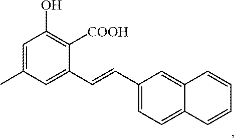 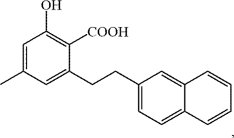 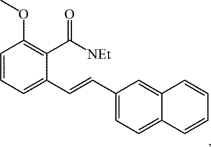 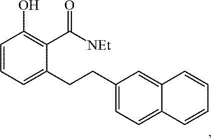 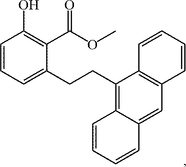 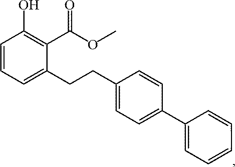 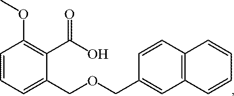 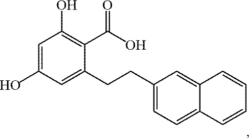 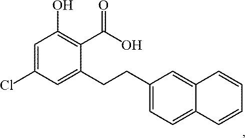 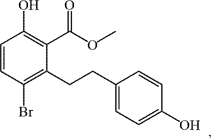 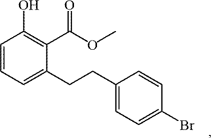 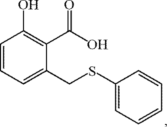 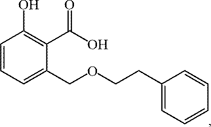 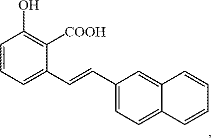 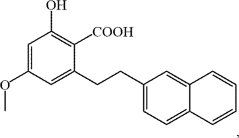 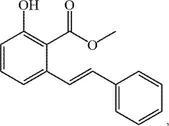 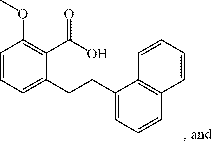 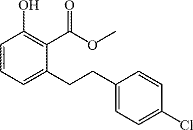 [SMILES: COC(=O)c1c(O)cccc1CCc1ccc(Cl)cc1]
.

13. The method according to claim 1, wherein the viral disease or condition is SOX18-dependent.

14. The method according to claim 1, wherein the compound selectively inhibits SOX18 activity.

15. The method according to claim 14, wherein the SOX18 activity includes contacting and/or binding to a DNA and/or protein sequence selected from the group consisting of RBPJ, SOX7, XRCC5, SOX18, ILF3 and DDX17.

16. The method according to claim 14, wherein the compound of Formula 1 inhibits SOX18 homodimerisation or SOX18-RBPJ heterodimersation.

17. (canceled)

18. The method according to claim 1 wherein the viral disease or condition is caused by a herpesvirus.

19. The method according to claim 18, wherein the Herpesvirus is selected from the group consisting of Alpha Herpesviruses, Beta Herpesviruses, Gamma₁ Herpesviruses and Gamma₂ Herpesviruses.

20. The method according to claim 18, wherein the herpesvirus is selected from the group consisting of Kaposi sarcoma herpesvirus (KSHV), Epstein-Barr virus (EBV), cytomegalovirus (CMV), Vesicular stomatitis virus (VSV), rhesus lymocryptovirus (rLCV), Herpes simplex virus 1 (HSV-1), Herpes simplex virus (HSV-2) and Ross river virus (RRV).

21. The method according to claim 1, wherein the viral disease or condition is selected from the group consisting of:
(i) Kaposi sarcoma caused by KSHV;
(ii) AIDs-related lymphoproliferative disorder caused by KSHV;
(iii) Angio-immunoblastic T-cell lymphoma caused by EBV;
(iv) Burkitt’s lymphoma caused by EBV;
(v) Hodgkin’s lymphoma caused by EBV;
(vi) Non-Hodgkin’s lymphoma caused by EBV;
(vii) Leiomyosarcoma caused by EBV;
(viii) Breast cancer caused by EBV;
(ix) Gastric carcinoma caused by EBV
(x) Nasal T/NK cell lymphoma caused by EBV;
(xi) T/NK cell lymphoma caused by EBV;
(xii) Nasopharyngeal carcinoma caused by EBV
(xiii) CMV retinitis of the eyes caused by CMV;
(xiv) Pneumonia caused by CMV;
(xv) Gastrointestinal ulcers caused by CMV;
(xvi) Condition or symptom caused by VSV;
(xvii) Oral herpes caused by HSV-1;
(xviii) Genital herpes caused by HSV-2; and
(xix) Joint inflammation or rash caused by RRV.

22. The method according to claim 1, wherein the compound is administered in the form of a pharmaceutical composition comprising a pharmaceutically acceptable excipient.

23-24. (canceled)
